(19) United States
(12) Patent Application Publication
(10) Pub. No.: US 2021/0322730 A1
(43) Pub. Date: Oct. 21, 2021

(54) GUIDEWIRE HAVING BONDED PROXIMAL AND DISTAL SEGMENTS

(71) Applicant: ABBOTT CARDIOVASCULAR SYSTEMS INC., SANTA CLARA, CA (US)

(72) Inventor: Robert Charles Hayzelden, Murrieta, CA (US)

(73) Assignee: ABBOTT CARDIOVASCULAR SYSTEMS INC., SANTA CLARA, CA (US)

(21) Appl. No.: 16/853,468

(22) Filed: Apr. 20, 2020

(51) Int. Cl. A61M 25/09; A61M 25/01

(52) U.S. Cl. CPC A61M 25/09; A61M 25/0138; A61M 2025/09058; A61M 2025/09108; A61M 2025/09141

(57) ABSTRACT

A guidewire having a proximal core wire formed from a first metal alloy is connected to a distal core wire formed from a second metal alloy. A tubular member is sized to receive an end of the proximal core wire and an end of the distal core wire in a butting configuration. The tubular member is attached to the proximal core wire and the distal core wire to form a joint connecting the two wires together.

### BACKGROUND

Conventional guidewires for angioplasty and other vascular procedures usually comprise an elongated core member with one or more tapered sections near the distal end thereof and a flexible body such as a helical coil disposed about the distal portion of the core member. A shapeable member, which may be the distal extremity of the core member or a separate shaping ribbon which is secured to the distal extremity of the core member, extends through the flexible body and is secured to a rounded plug at the distal end of the flexible body. Torquing means are provided on the proximal end of the core member to rotate, and thereby steer, the guidewire while it is being advanced through a patient's vascular system.

In a typical PTCA procedure, a guiding catheter having a preformed distal tip is percutaneously introduced into the cardiovascular system of a patient in a known manner and advanced therein until the distal tip of the guiding catheter is seated in the ostium of a desired coronary artery. A guidewire is positioned within an inner lumen of a dilatation catheter and then both are advanced through the guiding catheter to the distal end thereof. The guidewire is first advanced out of the distal end of the guiding catheter into the patient's coronary vasculature until the distal end of the guidewire crosses a lesion to be dilated, then the dilatation catheter having an inflatable balloon on the distal portion thereof is advanced into the patient's coronary anatomy over the previously introduced guidewire until the balloon of the dilatation catheter is properly positioned across the lesion. Once in position across the lesion, the balloon is inflated to a predetermined size with radiopaque liquid at relatively high pressures (e.g., greater than 4 atmospheres) to press the arteriosclerotic plaque of the lesion against the inside of the artery wall and to otherwise expand the inner lumen of the artery. The balloon is then deflated so that blood flow is resumed through the dilated artery and the dilatation catheter can be removed therefrom. Typically, the guidewire is left in the vasculature for further procedures, such as stenting.

A major requirement for guidewires is that they have sufficient column strength to be pushed through a patient's vascular system or other body lumen without kinking. However, they must also be flexible enough to avoid damaging the blood vessel or other body lumen through which they are advanced. Efforts have been made to improve both the strength and flexibility of guidewires to make them more suitable for their intended uses, but these two properties are for the most part diametrically opposed to one another in that an increase in one usually involves a decrease in the other.

Traditional guidewire construction consists of a stainless steel material, core to tip, with the distal (tip) ground to various profiles determined by the needs of the interventional case at hand. In some cases, a more complaint distal segment constructed of different materials are utilized in place of stainless steel to navigate through the tortuous anatomy. A typical alternative to stainless steel is a distal section constructed of nitinol which works well for the application, but is challenging to achieve a reliable bond due to material properties. The present invention solves these problems by providing a guidewire having a proximal section that is torqueable and is joined to a distal section that is highly flexible.

### SUMMARY OF THE INVENTION

In one embodiment, a guidewire is comprised of a proximal core wire having a proximal end and a distal end and being formed from a first metal alloy. A distal core wire has a proximal end and a distal end and is formed form a second metal alloy, different from the first metal alloy. A tubular member has a proximal end and a distal end and a lumen extending therethrough, and is formed from the first metal alloy. The lumen of the tubular member is sized to receive the distal end of the proximal core wire and the proximal end of the distal core wire in a butting configuration. The tubular member is attached to the distal end of the proximal core wire and to the proximal end of the distal end core wire. The attachment can be any suitable means such as by welding, brazing, bonding, and adhesive bonding. Preferably, the attachment is made by laser welding. In one embodiment, the first metal alloy is stainless steel and the second metal alloy is a superelastic material, such as nitinol. In one embodiment, a plurality of slits are formed in the tubular member to increase compliance and flexibility in the tubular member where the proximal core wire and the distal core wire are joined together.

In one embodiment, a guidewire is comprised of a proximal core wire having a proximal end and a distal end and being formed from a first metal alloy. A distal core wire has a proximal end and a distal end and is formed form a second metal alloy, different from the first metal alloy. A tubular member has a proximal end and a distal end and a lumen extending therethrough, and is formed from the first metal alloy. The lumen of the tubular member is sized to receive the distal end of the proximal core wire and the proximal end of the distal core wire in a spaced apart configuration. The tubular member is attached to the distal end of the proximal core wire and to the proximal end of the distal end core wire. The attachment can be any suitable means such as by welding, brazing, bonding, and adhesive bonding. Preferably, the attachment is made by laser welding. In one embodiment, the first metal alloy is stainless steel and the second metal alloy is a superelastic material, such as nitinol. In one embodiment, a plurality of slits are formed in the tubular member to increase compliance and flexibility in the tubular member where the proximal core wire and the distal core wire are joined together.

In one embodiment, a guidewire is comprised of a proximal core wire having a proximal end and a distal end and being formed from a first metal alloy. A distal core wire has a proximal end and a distal end and is formed form a second metal alloy, different from the first metal alloy. A tubular member has a proximal end and a distal end and a lumen extending therethrough, and is formed from the first metal alloy. The outer diameter of the proximal core wire and the distal core wire are the same as the outer diameter of the tubular member. A distal section of the proximal core wire and a proximal end of the distal core wire have a reduced outer diameter. The lumen of the tubular member is sized to receive the reduced diameter distal section of the proximal core wire and the reduced diameter proximal section of the distal core wire in a butting configuration. The tubular member is attached to the proximal core wire and to the distal end core wire so that there is a uniform outer diameter where the proximal and distal core wires are joined to the tubular member. The attachment can be any suitable means such as by welding, brazing, bonding, and adhesive bonding. Preferably, the attachment is made by laser welding. In one embodiment, the first metal alloy is stainless steel and the second metal alloy is a superelastic material, such as nitinol.

In another embodiment, a guidewire is comprised of a proximal core wire having a proximal end and a distal end and being formed from a first metal alloy. A distal core wire has a proximal end and a distal end and is formed form a second metal alloy, different from the first metal alloy. A tubular member has a proximal end and a distal end and a lumen extending therethrough, and is formed from the first metal alloy. The outer diameter of the proximal core wire and the distal core wire are the same as the outer diameter of the tubular member. A distal section of the proximal core wire and a proximal end of the distal core wire have a reduced outer diameter. The lumen of the tubular member is sized to receive the reduced diameter of the distal section of the proximal core wire and the reduced diameter of the proximal section of the distal core wire in a butting configuration. The tubular member is attached to the proximal core wire and to the distal end core wire so that there is a uniform outer diameter where the proximal and distal core wires are joined to the tubular member. The attachment in this embodiment is by crimping the outer tubular member onto the reduced diameter sections. In one embodiment, the first metal alloy is stainless steel and the second metal alloy is a superelastic material, such as nitinol.

### BRIEF DESCRIPTION OF THE DRAWINGS

FIG. 1 is a side elevational view, in longitudinal cross section, depicting an exploded view of the guidewire comprising a proximal core wire, a tubular member, and a distal core wire prior to being assembled.

FIG. 2A is a side elevational view, in longitudinal cross section, of the guidewire of FIG. 1 where the proximal core wire and the distal core wire are inserted in the tubular member and joined together.

FIG. 2B is a side elevational view of the guidewire of FIG. 1 depicting the tubular member having slits extending longitudinally, circumferentially, or spiral shaped, to enhance flexibility.

FIG. 3 is a partial side elevational view, in longitudinal cross section, depicting the tubular member forming a joint between the proximal core wire and the distal core wire.

FIG. 4 is a partial side elevational view, in longitudinal cross section, depicting the bonding joints to bond the tubular member and the proximal core wire and distal core wire together.

FIG. 5 is a side elevational view, in longitudinal cross section, depicting an embodiment wherein the proximal core wire and the distal core wire are inserted in the tubular member and spaced apart prior to attachment.

FIG. 6 is a side elevational view depicting an embodiment wherein a proximal core wire and a distal core wire are inserted in a tubular member and mechanically attached such as by crimping.

FIG. 7 is a side elevational view depicting a proximal core wire and a distal core wire comprised of a coil member inserted in a tubular member and attached together.

FIG. 8 is a side elevational view, in longitudinal cross section, depicting a proximal core wire and a distal core wire inserted into a tubular member wherein the tubular member ends have been ground down to form a taper for a smooth transition.

FIG. 9A is a side elevational view of a guidewire having a proximal core wire and a distal core wire, and a tubular member having a lumen for receiving the distal core wire.

FIG. 9B is a side elevational view, in longitudinal cross section, depicting the guidewire of FIG. 9A wherein the proximal core wire has been attached to the tubular member in a butting configuration.

FIG. 9C is a side elevational view, in longitudinal cross section, depicting the guidewire of FIGS. 9A and 9B wherein the distal core wire has been inserted in a lumen of the tubular member and attached thereto to form a bond joint.

FIG. 10 is a side elevational view, in longitudinal cross-section, depicting a reduced section on both of a proximal core wire and a distal core wire for insertion into a tubular member.

FIG. 11 is a side elevational view, in longitudinal cross-section, depicting the reduced sections of the proximal core wire and the distal core wire of FIG. 10 inserted into the tubular member in a butting configuration and the tubular member welded to the proximal core wire and the distal core wire.

FIG. 12A is a side elevational view, in longitudinal cross-section, depicting a reduced section on both of a proximal core wire and a distal core wire for insertion into a tubular member.

FIG. 12B is a side elevational view, in longitudinal cross-section, depicting the reduce sections of the proximal core wire and the distal core wire inserted into the tubular member and the tubular member being crimped onto the reduced sections.

FIG. 13A is a side elevational view depicting a guidewire wire formed from a spiral cut tubing.

FIG. 13B is a transverse cross-sectional view taken along lines 13B-13B, depicting the guidewire of FIG. 13A.

### DETAILED DESCRIPTION OF THE PREFERRED EMBODIMENTS

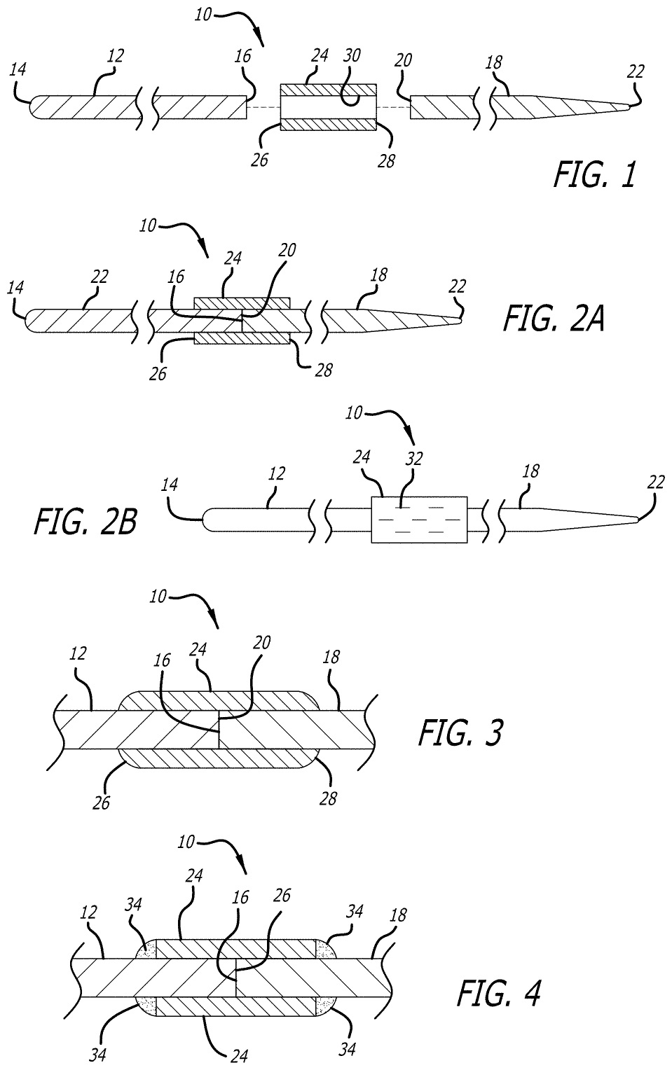

It is often desirable in PTCA procedures to utilize a guidewire having a high degree of torque and column strength in the proximal section, and be more compliant and flexible in the distal section to navigate tortuous coronary arteries. In one embodiment, shown in FIGS. 1-4, a guidewire 10 includes a proximal core wire 12 having a proximal end 14 and a distal end 16 and being formed from a first metal alloy. A distal core wire 18 has a proximal end 20 and a distal end 22 and is formed from a second metal alloy, different from the first metal alloy. A tubular member 24 has a proximal end 26 and a distal end 28 and a lumen 30 extending therethrough, and is formed from the first metal alloy. In conventional coronary guidewires, the proximal core wire and at least a portion of the distal core wire have a nominal outer diameter of 0.014 inch or 0.018 inch. Thus the lumen 30 of the tubular member 24 is slightly larger than the nominal outer diameter of the guidewire so that when the proximal core wire and the distal core wire are inserted into the lumen, there is a tight fit. The lumen 30 of the tubular member 24 is sized to receive the distal end 16 of the proximal core wire 12 and the proximal end 20 of the distal core wire 18 in a butting configuration. The tubular member 24 is attached to the distal end 16 of the proximal core wire 12 and to the proximal end 20 of the distal core wire 18 by any suitable means such as by welding, brazing, bonding, and adhesive bonding. Preferably, the attachment is made by laser welding 34 as shown in FIG. 4. In one embodiment, the first metal alloy is stainless steel and the second metal alloy is a superelastic material, such as nitinol. In one embodiment, shown in FIG. 2B, a plurality of slits 32 are formed in the tubular member 24 to increase compliance and flexibility in the tubular member where the proximal core wire 12 and the distal core wire 18 are joined together. The slits 32 can extend longitudinally as shown in FIG. 2B, or circumferentially or in a spiral direction.

In another embodiment, shown in FIG. 5, a guidewire 10 includes a proximal core wire 12 having a proximal end 14 and a distal end 16 and being formed from a first metal alloy. A distal core wire 18 has a proximal end 20 and a distal end 22 and is formed form a second metal alloy, different from the first metal alloy. A tubular member 24 has a proximal end 26 and a distal end 28 and a lumen 30 extending therethrough, and is formed from the first metal alloy. The lumen 30 of the tubular member is sized to receive the distal end 16 of the proximal core wire 12 and the proximal end 20 of the distal core wire 18 in a spaced apart configuration. Lumen 30 is similarly sized to that disclosed in FIGS. 1-4. The tubular member 24 is attached to the distal end 16 of the proximal core wire 12 and to the proximal end 20 of the distal end core wire 18. The attachment can be any suitable means such as by welding, brazing, bonding, and adhesive bonding. Preferably, the attachment is made by laser welding 34. In one embodiment, the first metal alloy is stainless steel and the second metal alloy is a superelastic material, such as nitinol.

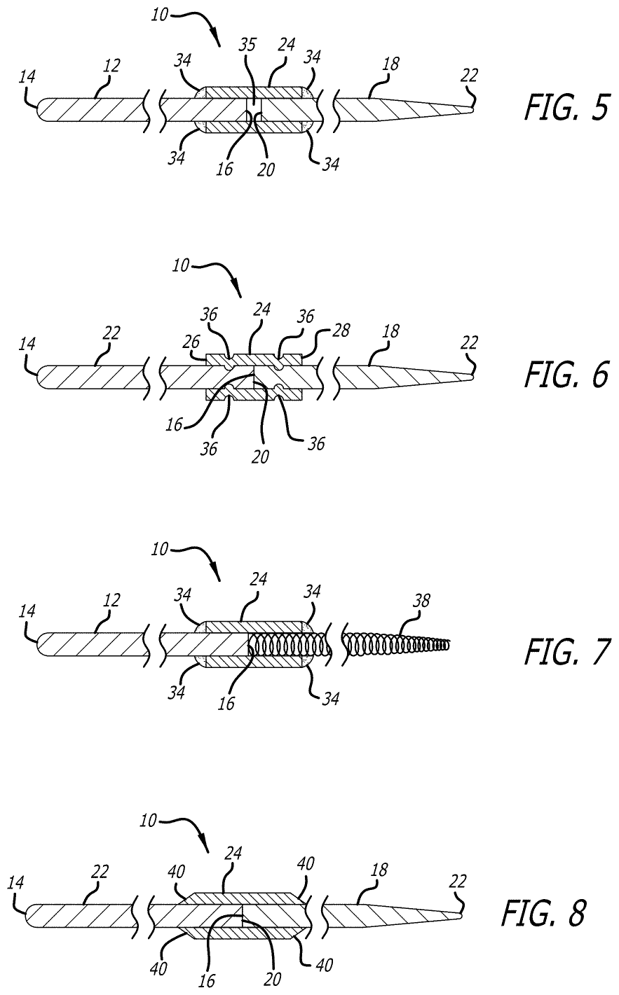

In the embodiment shown in FIG. 6, the guidewire 10 is the same as that described for FIGS. 1-4, except for the attachment means. In FIG. 6, the tubular member 24 is attached to the proximal core wire 12 and the distal core wire 18 by multiple crimps 36. The crimps 36 can be formed by any known means.

In the embodiment shown in FIG. 7, the guidewire is the same as that described for FIGS. 1-5, except for the distal core wire. In this embodiment, the distal core wire is a distal coil segment 38, preferably tapered, and inserted into the distal end 28 of the tubular member 24 and attached by any of the aforementioned bonding techniques, such as by laser welding 34. The distal coil segment 38 can include any configuration of coils including wound coils, multifilar coils, counter wound coils and tapered coils.

As shown in FIG. 8, the proximal end 26 and the distal end 28 of the tubular member 24 can be ground down to form a transition taper 40 onto the proximal core wire 12 and the distal core wire 16. Further, if the laser welding 34 or other bonding means form a metal or adhesive bulge, the grinding procedure will also form the transition taper 40. The transition taper 40 does not increase stiffness in the joint and provides a smooth outer surface where the proximal core wire 12 and the distal core wire 18 are attached to the tubular member 24. The transition taper 40 can be applied to any of the embodiments disclosed herein.

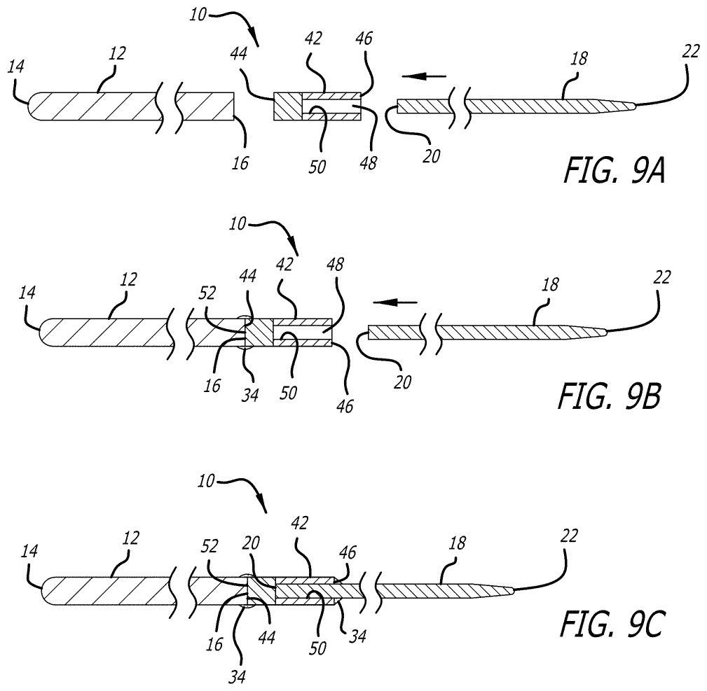

In the embodiment shown in FIGS. 9A-9C, a guidewire 10 includes a proximal core wire 12 having a proximal end 14 and a distal end 16 and being formed from a first metal alloy. A distal core wire 18 has a proximal end 20 and a distal end 22 and is formed from a second metal alloy, different from the first metal alloy. A tubular member 42 has a proximal end 44 and a distal end 46 and a lumen 48 extending partially therethrough from the distal end 46 toward the proximal end. The lumen 48 forms a receptacle 50 for receiving the proximal end 20 of the distal core wire 18. The tubular member 42 is formed from the first metal alloy. The receptacle 50 of the tubular member 42 is sized to receive the proximal end 20 of the distal core wire 18 in a tight fit configuration as previously described. The proximal end 44 of the tubular member 42 is attached to the distal end 16 of the proximal core wire 12 in a butting configuration 52. The attachment can be any suitable means such as by welding, brazing, bonding, adhesive bonding, crimping or swaging. Preferably, the attachment is made by laser welding 34. In one embodiment, the first metal alloy is stainless steel and the second metal alloy is a superelastic material, such as nitinol. As shown in FIGS. 9B and 9C, the proximal end 20 of the distal core wire 18 is inserted into the receptacle 50 and attached as described above, and preferably by laser welding 34. Importantly, the receptacle is configured to receive any type of distal segment configuration including, but not limited to, a solid core wire, laser cut hollow tubing, wound coils, hybrid coil/solid core, round or alternate profile coils (i.e., transverse cross-section coil having round, square, rectangular, I-beam, vertical rectangle and H-shaped configurations), multifilar coils, counter would coils and braided segments.

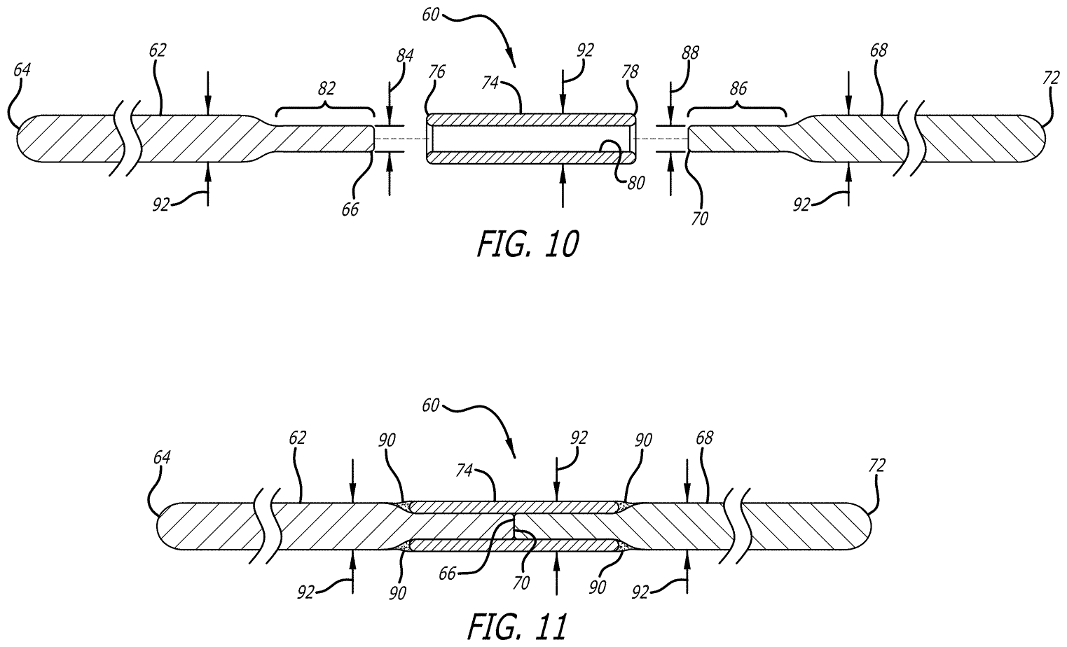

In another embodiment, shown in FIGS. 10 and 11, a guidewire 60 includes a proximal core wire 62 having a proximal end 64 and a distal end 66 and being formed from a first metal alloy. A distal core wire 68 has a proximal end 70 and a distal end 72 and is formed from a second metal alloy, different from the first metal alloy. A tubular member 74 has a proximal end 76 and a distal end 78 and a lumen 80 extending therethrough, and is formed from the first metal alloy. In conventional coronary guidewires, the proximal core wire and at least a portion of the distal core wire have a nominal outer diameter of 0.014 inch or 0.018 inch. In this embodiment, however, a distal section 82 of the proximal core wire 62 has a reduced outer diameter 84 and a proximal section 86 of the distal core wire 68 has a reduced outer diameter 88. Thus, the lumen 80 of the tubular member 74 is slightly larger than the reduced outer diameter 84 and the reduced outer diameter 88 so that when the proximal core wire 62 and the distal core wire 68 are inserted into the lumen 80, there is a tight fit. The lumen 80 of the tubular member 74 is sized to receive the reduced outer diameter 84 of the distal section 82 and the reduced outer diameter 88 of the proximal section 86 in a butting configuration. Importantly, because of the reduced outer diameters 84, 88 of the distal section 82 and the proximal section 86 respectively, there is a uniform outer diameter 92 where the proximal core wire 62, the distal core wire 68, and the tubular member 74 are joined. The tubular member 74 is attached to the distal section 82 of the proximal core wire 62 and to the proximal section 86 of the distal core wire 68 by any suitable means such as by welding, brazing, bonding, and adhesive bonding. Preferably, the attachment is made by laser welding 90 as shown in FIG. 11. In one embodiment, the first metal alloy is stainless steel and the second metal alloy is a superelastic material, such as nitinol.

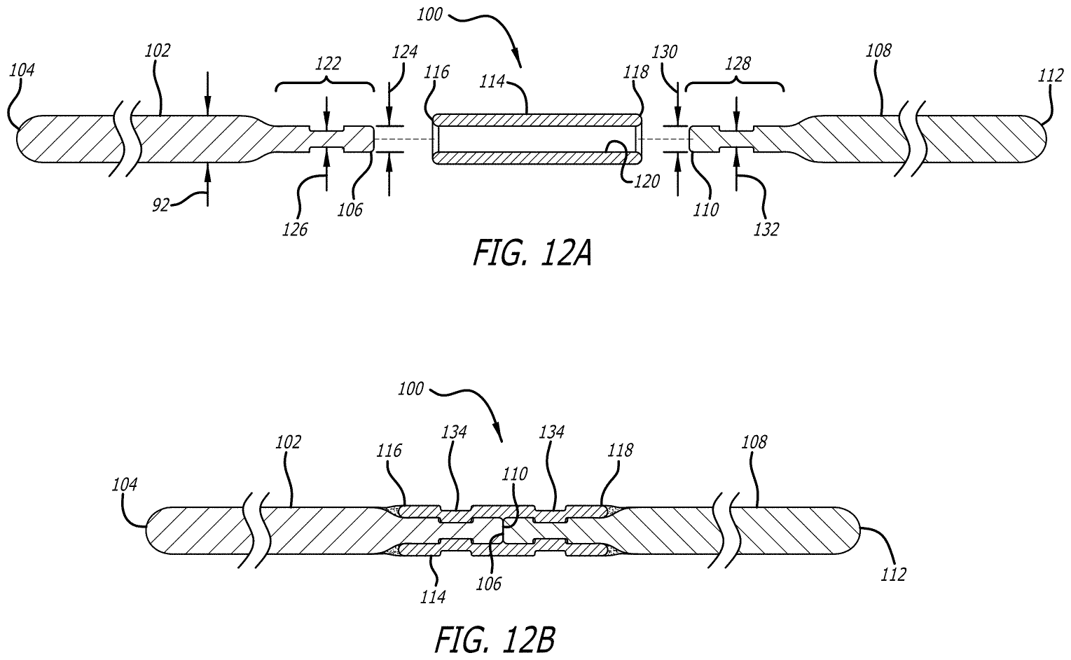

In one embodiment, shown in FIGS. 12A and 12B, a guidewire 100 includes a proximal core wire 102 having a proximal end 104 and a distal end 106 and being formed from a first metal alloy. A distal core wire 108 has a proximal end 110 and a distal end 112 and is formed from a second metal alloy, different from the first metal alloy. A tubular member 114 has a proximal end 116 and a distal end 118 and a lumen 120 extending therethrough, and is formed from the first metal alloy. In conventional coronary guidewires, the proximal core wire and at least a portion of the distal core wire have a nominal outer diameter of 0.014 inch or 0.018 inch. In this embodiment, however a distal section 122 of the proximal core wire 102 has a first reduced outer diameter 124 and a second reduced outer diameter 126. Further, a proximal section 128 of the distal core wire 108 has a first reduced outer diameter 130 and a second reduced outer diameter 132. Thus, the lumen 120 of the tubular member 114 is slightly larger than the first reduced outer diameter 124, 130 so that when the proximal core wire 102 and the distal core wire 108 are inserted into the lumen 120, there is a tight fit. The lumen 120 of the tubular member 114 is sized to receive the first reduced outer diameter 124 of the proximal core wire 102 and the first reduced outer diameter 130 of the distal core wire 108 in a butting configuration. As shown in FIG. 12B, the tubular member 114 is attached to the first reduced outer diameter 124 of the proximal core wire 102 and to the first reduced outer diameter 130 of the distal core wire 108 by forming a crimp 134 in the tubular member 114 to coincide with the second reduced outer diameter 126 of the proximal core wire 102 and with the second reduced outer diameter 132 of the distal core wire 108. Alternatively, swaging can be used instead of crimping. In one embodiment, the first metal alloy is stainless steel and the second metal alloy is a superelastic material, such as nitinol.

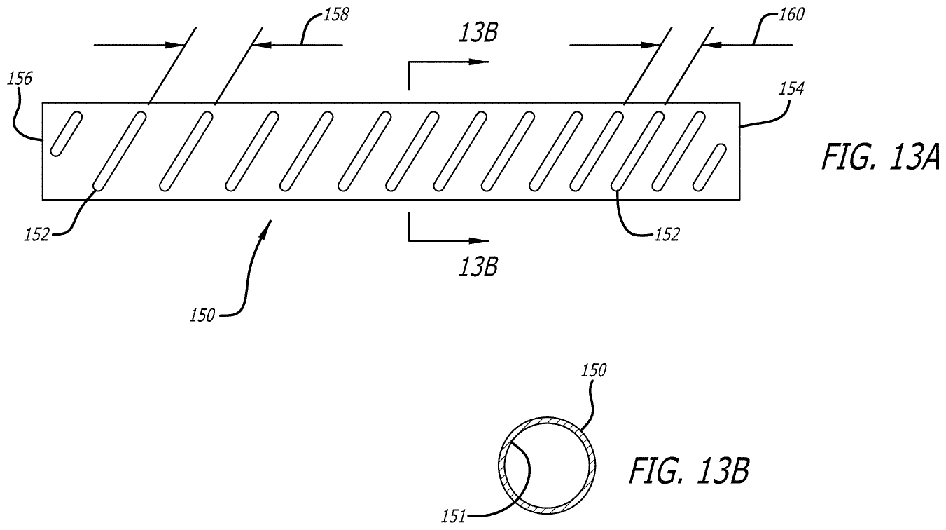

In another embodiment, shown in FIGS. 13A and 13B, the guidewire is the same as that described for FIGS. 1-5 and 7, except for the distal section of the guidewire. In this embodiment, the distal section 150 is a tubular member having a lumen 151 and a plurality of slits 152 to add flexibility to the distal section 150, which replaces a distal coil end segment 138 such as shown in FIG. 7. The distal section 150 has a distal end 154 and a proximal end 156 configured for insertion into and attached to a tubular member 24 like the one shown in FIGS. 1-5 and 7. The number of slits 152 in the distal segment 150 can vary in size, shape, angularity and spacing in order to achieve a desired compliance. The distal segment 150 can be tapered (not shown) toward the distal end as is known in the art. In one embodiment, the slits 152 have a first spacing 158 at the proximal end 156 and a second spacing 160 at the distal end.

In all of the disclosed embodiments, portions of or all of the guidewires 10 may be coated with a polymer jacket in a known manner to enhance the smoothness of the outer surface and reduce friction as the guidewire is advanced through a guide tube and through tortuous vasculature.

While the description of embodiments having features of the invention has been directed primarily herein to guidewires suitable for guiding other devices within a patient's body, those skilled in the art will recognize that these features may also be utilized in other intracorporeal devices such as electrophysiology catheters, pacing leads and the like. References to other modifications and improvements can be made to the invention without departing from the scope of the appended claims.

To the extent not otherwise described herein, the materials and methods of construction and the dimensions of conventional intracorporeal devices such as intravascular guidewires may be employed with a device embodying features of the present invention. Moreover, features disclosed with one embodiment may be employed with other described embodiments.

I claim:

1. A guidewire, comprising:
a proximal core wire having a proximal end and a distal end and formed from a first metal alloy;
a distal core wire having a proximal end and a distal end and formed from a second metal alloy different from the first metal alloy;
a tubular member having a proximal end, a distal end, a lumen extending therethrough, and formed from the first metal alloy;
wherein the lumen of the tubular member is sized to receive the distal end of the proximal core wire and the proximal end of the distal core wire in a butting configuration; and
attaching the tubular member to the distal end of the proximal core wire and to the proximal end of the distal core wire.

2. The guidewire of claim 1, wherein the first metal alloy is stainless steel and the second metal alloy is a superelastic metal alloy.

3. The guidewire of claim 2, wherein the superelastic metal alloy is nitinol.

4. The guidewire of claim 3, wherein the tubular member has a radiused proximal end and distal end to provide a smooth transition with the proximal core wire and the distal core wire.

5. The guidewire of claim 3, wherein the proximal end and the distal end of the tubular member are ground down to provide a smooth transition section with the proximal core wire and the distal core wire.

6. The guidewire of claim 1, wherein the proximal core wire and the distal core wire are attached to the tubular member by laser welding, bonding, adhesive bonding or brazing.

7. The guidewire of claim 6, wherein the proximal end and the distal end of the tubular member are ground down to provide a smooth transition section with the proximal core wire and the distal core wire.

8. The guidewire of claim 1, wherein a first weld bond joint attaches the proximal end of the tubular member to the proximal core wire and a second weld bond joint attaches the distal end of the tubular member to the distal core wire.

9. The guidewire of claim 1, wherein a plurality of slits are formed in the tubular member to increase compliance and flexibility.

10. The guidewire of claim 9, wherein the plurality of slits are formed by a laser.

11. A guidewire, comprising:
a proximal core wire having a proximal end and a distal end and formed from a first metal alloy;
a distal core wire having a proximal end and a distal end and formed from a second metal alloy different from the first metal alloy;
a tubular member having a proximal end a distal end, a lumen extending therethrough, and formed from the first metal alloy;
wherein the lumen of the tubular member is sized to receive the distal end of the proximal core wire and the proximal end of the distal core wire so that the proximal end is spaced apart from the distal end; and
attaching the tubular member to the distal end of the proximal core wire and to the proximal end of the distal core wire.

12. The guidewire of claim 11, wherein the first metal alloy is stainless steel and the second metal alloy is a superelastic metal alloy.

13. The guidewire of claim 12, wherein the superelastic metal alloy is nitinol.

14. The guidewire of claim 13, wherein the tubular member has a radiused proximal end and distal end to provide a smooth transition with the proximal core wire and the distal core wire.

15. The guidewire of claim 13, wherein the proximal end and the distal end of the tubular member are ground down to provide a smooth transition section with the proximal core wire and the distal core wire.

16. The guidewire of claim 11, wherein the proximal core wire and the distal core wire are attached to the tubular member by laser welding, bonding, adhesive bonding or brazing.

17. The guidewire of claim 11, wherein a first weld bond joint attaches the proximal end of the tubular member to the proximal core wire and a second weld bond joint attaches the distal end of the tubular member to the distal core wire.

18. The guidewire of claim 11, wherein a plurality of slits are formed in the tubular member to increase compliance and flexibility.

19. A guidewire, comprising:
a proximal core wire having a proximal end and a distal end and formed from a first metal alloy;
a distal core wire having a proximal end and a distal end and formed from a second metal alloy different from the first metal alloy;
a tubular member having a proximal end, a distal end, a lumen extending a distance from the distal end, and formed from the first metal alloy;
wherein the distal end of the proximal core wire is attached to the proximal end of the tubular member in a butting configuration;
the lumen of the tubular member being sized to receive the proximal end of the distal core wire; and
attaching the tubular member to the proximal end of the distal core wire.

20. The guidewire of claim 19, wherein the first metal alloy is stainless steel and the second metal alloy is a superelastic metal alloy.

21. The guidewire of claim 20, wherein the superelastic metal alloy is nitinol.

22. The guidewire of claim 21, wherein the tubular member has a radiused distal end to provide a smooth transition with the distal core wire.

23. The guidewire of claim 21, wherein the distal end of the tubular member is ground down to provide a smooth transition section with the distal core wire.

24. The guidewire of claim 19, wherein the proximal core wire and the distal core wire are attached to the tubular member by laser welding, bonding, adhesive bonding or brazing.

25. The guidewire of claim 19, wherein a first weld bond joint attaches the proximal end of the tubular member to the proximal core wire and a second weld bond joint attaches the distal end of the tubular member to the distal core wire.

26. The guidewire of claim 19, wherein a plurality of slits are formed in the tubular member to increase compliance and flexibility.

27. The guidewire of claim 19, wherein the lumen of the tubular member provides a receptacle to accommodate any configuration of distal core wire including solid core wire, laser cut tubing, wound coils, hybrid coil/solid core wire, multifilar coils, counter wound coils, and braided wires.
